(19) United States
(12) Patent Application Publication
(10) Pub. No.: US 2025/0049313 A1
(43) Pub. Date: Feb. 13, 2025

(54) ELECTRONIC ASSEMBLIES FOR MEDICAL DEVICES AND ASSOCIATED METHODS

(71) Applicant: Boston Scientific Scimed, Inc., Maple Grove, MN (US)

(72) Inventors: Richard C. TAH, Milford, MA (US); Eric Noble LENT, Roberts, WI (US); Vivek SHAH, Burlington, MA (US); James E. BLOOD, Shoreview, MN (US); Dennis LYNCH, Co. Cork (IE); Gilles PLICHON, Marlborough, MA (US)

(73) Assignee: Boston Scientific Scimed, Inc., Maple Grove, MN (US)

(21) Appl. No.: 18/795,388

(22) Filed: Aug. 6, 2024

Related U.S. Application Data
(60) Provisional application No. 63/517,991, filed on Aug. 7, 2023.

(51) Int. Cl. A61B 1/307; A61B 1/00; A61B 1/05; A61B 1/06

(52) U.S. Cl. CPC A61B 1/307; A61B 1/00066; A61B 1/00097; A61B 1/00124; A61B 1/05; A61B 1/0684

(57) ABSTRACT

Electronic assemblies for medical devices are described, including an electronic assembly that includes a body, at least one imaging device, and at least one light source. The body may include a plurality of electrical connection points and a plurality of electrical traces in a material that is electrically insulative. The plurality of electrical connection points may be on one or more external surfaces of the body. The plurality of electrical connection points may include proximal and distal connection points. Each electrical trace may connect a proximal connection point to a respective distal connection point. The at least one imaging device may be coupled to one or more of the distal connection points and/or the at least one light source may be coupled to one or more of the distal connection points.

### CROSS-REFERENCE TO RELATED APPLICATIONS

This application claims the benefit of priority to U.S. Provisional Application No. 63/517,991, filed on Aug. 7, 2023, which is incorporated by reference herein in its entirety.

### TECHNICAL FIELD

This disclosure relates generally to electronic assemblies for medical devices and associated methods. More specifically, aspects of this disclosure pertain to assemblies including components that are positioned at a distal tip of a medical device, such as an endoscope

### BACKGROUND

In a medical procedure, an operator may insert a medical device, such as an endoscope or other type of scope, into a body lumen of a subject. The operator may navigate the distal tip to a location where the procedure is to be performed. The operator may pass accessory devices (e.g., instruments) through a working channel of the medical device to assist with performing certain diagnostic or therapeutic procedures. The distal tip of the medical device may include elements for providing visualization of the body lumen, such as lighting elements and/or imaging devices. The distal tip of the medical device also may include other features, such as an opening of the working channel, one or more openings for air/water and suction, and/or an elevator. Many current scopes present limitations on the arrangement of elements and/or types of elements that may be incorporated into the distal tip of the device. It may be desirable to increase a size of the working channel and its associated opening or introduce additional elements to the distal tip of the medical device without increasing an outer diameter of the medical device. It may further be desirable to deliver images of high quality to an operator.

### SUMMARY

Each of the aspects disclosed herein may include one or more of the features described in connection with any of the other disclosed aspects.

In some aspects, an electronic assembly for a medical device may include a body, at least one imaging device, and at least one light source. The body may include a plurality of electrical connection points and a plurality of electrical traces in a material that is electrically insulative. The plurality of electrical connection points may be on one or more external surfaces of the body. The plurality of electrical connection points may include, e.g., proximal connection points and distal connection points. Each electrical trace of the plurality of electrical traces may connect a proximal connection point to a respective distal connection point. The at least one imaging device may be coupled to one or more of the distal connection points and/or the at least one light source may be coupled to one or more of the distal connection points.

The electronic assembly may include one or more of the following features. The at least one imaging device and the at least one light source may face in a same direction. In some examples, the plurality of electrical connection points may include a plurality of cable connection points and at least one sensor connection point. The electronic assembly may further include at least one sensor coupled to the body on a side of the body opposite at least one electrical connection point of the plurality of electrical connection points. The at least one sensor may include, for example, a pressure sensor. The body may include an end face and at least one arm adjacent to, and extending distally of, the end face. The end face may include at least one of the distal connection points, and the at least one imaging device may be coupled to the distal connection point on the end face. The at least one arm may include at least one of the distal connection points, and the at least one light source may be coupled to the distal connection point on the at least one arm.

The at least one light source may include two light-emitting diodes (LEDs) positioned on opposing sides of the at least one imaging device. The LEDs may be proximal to the at least one imaging device. The material of the body may include a ceramic and/or other non-conductive materials. The plurality of electrical traces may include copper and/or other conductive materials. The plurality of electrical traces may pass through an interior of the body. In some aspects of the present disclosure, the electronic assembly may further include a capacitor and/or a resistor. The body may include an end face that has a curved surface.

In one or more other aspects of the present disclosure, an electronic assembly for a medical device may include a body, at least one imaging device, and at least one light source. The body may include an end face and at least one arm adjacent to and extending distally of the end face. The body may include an electrically insulative material, a plurality of electrical connection points, and a plurality of electrically conductive traces. The at least one imaging device may be coupled to the end face of the body. The at least one light source may be coupled to the at least one arm.

The electronic assembly may include one or more of the following aspects. The plurality of electrical connection points may include proximal connection points and distal connection points. Each electrically conductive trace of the plurality of electrically conductive traces may electrically connect a proximal connection point to a respective distal connection point. The electronic assembly may further include a pressure sensor coupled to one or more of the electrical connection points of the plurality of electrical connection points.

Additionally, in one or more other aspects of the present disclosure, an electronic assembly for a medical device may include a body, at least one imaging device, and at least one light source. The body may include an end face and at least one arm adjacent to the end face. The body may include an electrically insulative material, a plurality of electrical connection points on an external surface of the body, and a plurality of electrically conductive traces that passes through an interior of the body between two electrical connection points of the plurality of electrical connection points. The at least one imaging device may be coupled to the end face. The at least one light source may be coupled to the at least one arm.

The electronic assembly may include one or more of the following aspects. The plurality of electrical connection points may include proximal connection points and distal connection points. A proximal portion of the body may include the proximal connection points, and an intermediate portion of the body may include the distal connection points. The body may include a stepped portion between the proximal portion and the intermediate portion.

### BRIEF DESCRIPTION OF THE DRAWINGS

The accompanying drawings, which are incorporated in and constitute a part of this application, illustrate examples of this disclosure and together with the description, serve to explain the principles of the disclosure.

FIGS. 1A and 1B depict an exemplary medical device.

FIGS. 2A and 2B depict an exemplary electronic assembly for use with a medical device, such as the medical device of FIGS. 1A and 1B.

FIGS. 3A-3F depict various views of an exemplary distal structure for an electronic assembly, such as the electronic assembly of FIGS. 2A and 2B.

FIG. 4 depicts a distal portion of another exemplary medical device.

FIGS. 5A-5D depict various view of another exemplary electronic assembly for use with a medical device, such as the medical device of FIG. 4

FIGS. 6A-6C depict various views of another exemplary distal structure for an electronic assembly, such as the electronic assembly of FIGS. 5A-5D.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The drawings may include arrows labeled “P” and “D,” indicating proximal and distal directions, respectively. The term “approximately,” or like terms (e.g., “substantially”), includes values+/−10% of a stated value.

A medical device, such as an ureteroscope may be inserted into a body lumen of a subject (e.g., into a kidney) in order to perform a medical procedure (e.g., a laser lithotripsy procedure). The distal tip of the medical device may include one or more electronic assemblies each including various components, such as illumination or lighting elements (e.g., light emitting diodes (LEDs), optical fibers (e.g., plastic optical fibers or POFs) or other light guides, or a combination thereof), imaging elements (e.g., cameras, other components having imagers, or other optical elements (e.g., lenses)), and/or associated electronic elements (e.g., capacitors, diodes, resistors, and the like). Electronic components of the assemblies herein may include electrical connections, and may also include various elements mounted, for example, or otherwise connected to the electrical connections.

It may be desirable for electronic assemblies to occupy a relatively small or limited amount of space while retaining quality of performance and manufacturing. For example, with the electronic assembly (ies) occupying a small amount of space, the medical device may be capable of accommodating additional components, and/or having a larger (e.g., cross-sectional diameter or area) and/or additional working channel(s) or lumen(s). Additionally or alternatively, with the electronic assembly (ies) occupying a small amount of space, the medical device (e.g., a distal portion of the medical device) may include a smaller overall size (e.g., diameter or other cross-sectional size). One exemplary manner of conserving space while retaining quality of performance and manufacturing is to integrate one or more components (e.g., imaging device(s) such as camera(s), illumination device(s) such as LED(s), pressure and/or temperature sensor(s), etc.) into a distal tip or end cap of a medical device (e.g., an endoscope). As a result, in some aspects, a three-dimensional (3D) electronic assembly may be integrated into the distal tip or end cap, helping to allow for a reduced profile of the medical device (e.g., the shaft and/or distal end of the medical device). The smaller medical device may be able to access a desired anatomy, for example, due to the smaller insertion portion or distal end of the medical device, while still maintaining the capabilities (e.g., imaging, illumination, pressure and/or temperature sensing, working channel/lumens, etc.) of a larger medical device.

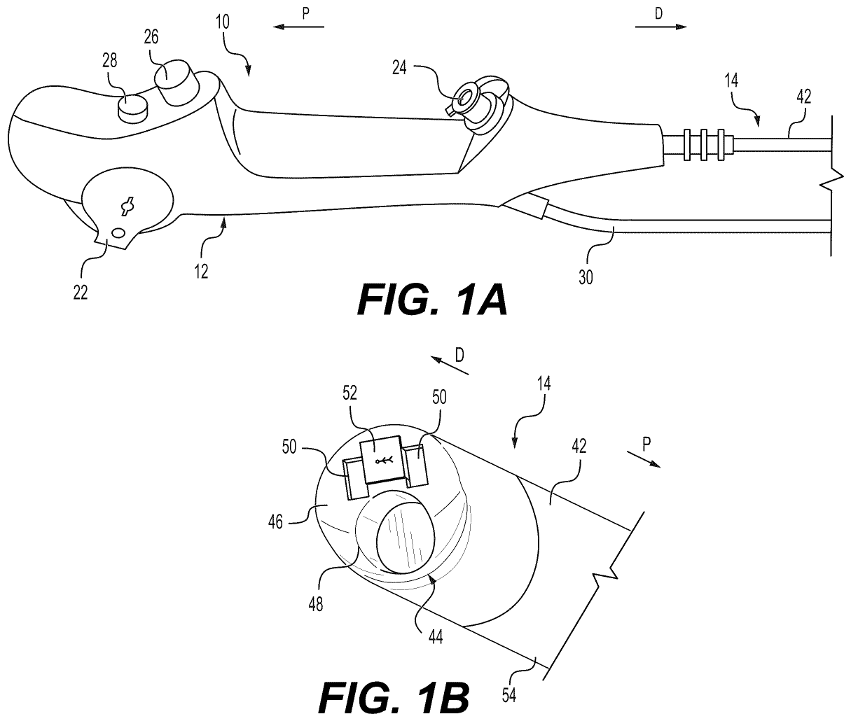

FIGS. 1A and 1B depict aspects of an exemplary medical device 10 according to the disclosure. FIG. 1A depicts a proximal portion of medical device 10. FIG. 1B depicts a distal tip 44 of medical device 10. Medical device 10 may include a handle portion 12 for gripping and use by an operator, and an insertion portion 14 for at least partial insertion into a body (e.g., a body lumen) of a subject. As discussed in detail below, insertion portion 14 (e.g., distal tip 44 of insertion portion 14) may include one or more electrical components, for example, electrically connected to one or more components of handle portion 12 through insertion portion 14. As shown in FIGS. 1A and 1B, medical device 10 may include a ureteroscope. Although the disclosure may refer at different points to a ureteroscope, it will be appreciated that, unless otherwise specified, endoscope, duodenoscopes, endoscopes, gastroscopes, endoscopic ultrasonography (“EUS”) scopes, colonoscopes, bronchoscopes, laparoscopes, cystoscopes, aspiration scopes, sheaths, catheters, or any other suitable medical device may be used in connection with the elements and assemblies described herein.

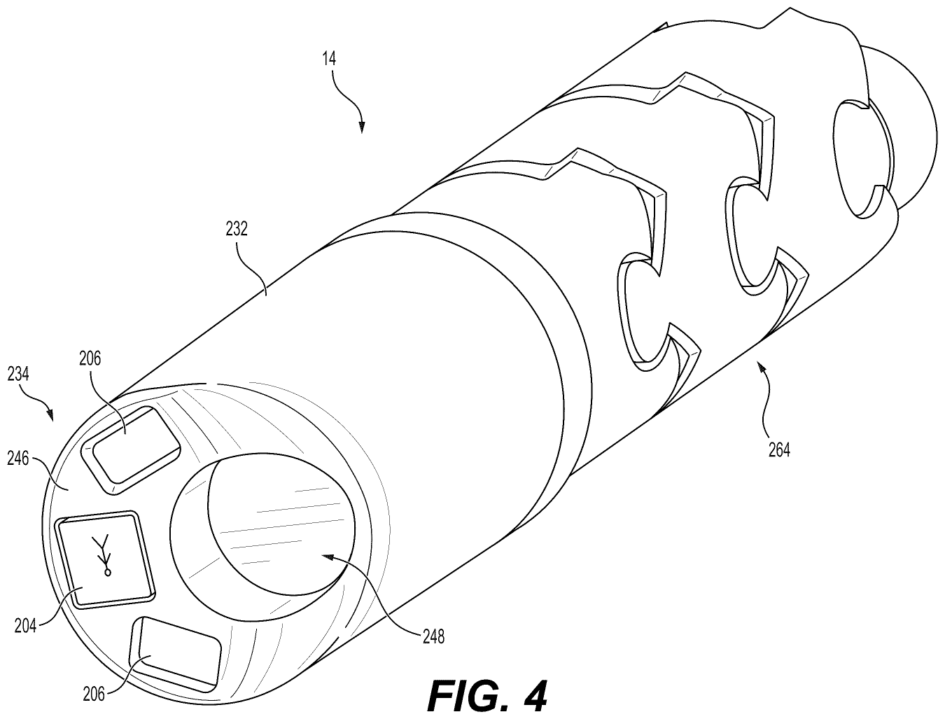

Handle portion 12 may include a control or actuation mechanism, e.g., lever 22. In some examples, lever 22 may be disposed on a proximal portion of handle portion 12. Lever 22 may help to facilitate articulation/steering of insertion portion 14, including distal tip 44. For example, a distal portion of insertion portion 14 may include an articulation portion 54, which may include one or more articulation joints (see, e.g., FIG. 4), for example, enclosed within an outer sheath of insertion portion 14. Although lever 22 is depicted in FIG. 1A, it will be appreciated that any suitable actuator(s) may be used in addition to or in place of lever 22, such as one or more knobs, buttons, sliders, switches, or joysticks. Moreover, handle portion 12 may include one or more locking mechanisms, for example, to lock a position of lever 22 and thus an orientation of a portion of insertion portion 14 (e.g., distal tip 44).

A port 24 of handle portion 12 may provide access to a lumen or working channel of medical device 10. An operator may insert an instrument or other device into port 24 and may extend the instrument or other device distally through the working channel. The working channel may extend longitudinally through a length of insertion portion 14. Handle portion 12 may also include a suction valve 26, for example, on a proximal portion of handle portion 12 and on an opposing side from lever 22. An operator may connect suction valve 26 to a source of suction, and may operate suction valve 26 to generate suction through insertion portion 14 (e.g., through a lumen or working channel, which may be the same or different from the lumen or working channel in communication with the port 24). Handle portion 12 may additionally or alternatively include other types of valves, e.g., fluid supply valves (e.g., air and/or water valves), or valves that perform a combination of functions.

An actuator such as image capture button 28 of handle portion 12 may enable an operator to capture a still image using an imaging device 52 (shown in FIG. 1B and described in further detail below) during a procedure. Additionally or alternatively, image capture button 28 may enable an operator to capture video or to perform other functions to control medical device 10. Image capture button 28 may be positioned on a proximal portion of handle portion 12, for example, adjacent suction valve 26 as shown, or another part of handle portion accessible to the operator's hand during use.

An umbilicus 30 may extend from handle portion 12 (e.g., from a distal portion of handle portion 12 as shown, or another part of handle portion 12) and may include or carry wires, cables, and/or conduits for providing, for example, power, signals, or fluids to and/or from handle portion 12. For example, umbilicus 30 may connect handle portion 12 to one or more user interfaces, monitors, control units, displays, etc.

Insertion portion 14 may include a shaft 42 extending distally from handle portion 12. At least part of shaft 42 may be flexible to facilitate navigation through anatomy, and shaft 42 may have wires, tubes, or other features passing therethrough. Distal tip 44 of medical device 10 may be disposed at a distal end of shaft 42. As shown in FIG. 1B, distal tip 44 may include a distalmost face 46. Distalmost face 46 may define a working channel opening 48. The working channel may extend between port 24 and working channel opening 48, such that instruments or other devices may be passed through port 24, through the working channel, and extend out of working channel opening 48. An instrument extending distally of working channel opening 48 may be used to perform a medical procedure on a subject.

Distal tip 44 may also include imaging components, such as one or more light sources 50 and one or more imaging devices 52 (e.g., camera(s)). Although two light sources 50 and one imaging device 52 are depicted in FIG. 1B, it will be appreciated that other combinations of light source(s) 50 and imaging device(s) 52 may be utilized. Alternatively, light sources 50 and imaging device 52 may be combined into a single unit or device. Light sources 50 may include LEDs, optical fibers (e.g., plastic optical fibers (POFs)), or any other suitable light source. Imaging device 52 may be configured to take video and/or still images. Imaging device 52 may provide a signal to a monitor or display (e.g., connected via umbilicus 30), so that an operator may view a visual image provided by imaging device 52 while navigating medical device 10 through a body lumen of a subject.

As depicted in FIG. 1B and described above, medical device 10 may be forward-facing. In other words, features of distal tip 44 (e.g., working channel opening 48, light sources 50, and imaging device 52) may face in a same direction, for example, a distal direction (i.e., facing distal/forward of distalmost face 46). This disclosure also encompasses other configurations of distal tip 44. For example, medical device 10 may be side-facing. In a side-facing example, working channel opening 48, light sources 50, and/or imaging device 52 may be disposed on a side of distal tip 44 or otherwise incorporated into distal tip 44 so that they point in a radially outward direction, relative to a longitudinal axis of insertion portion 14. In some examples, medical device 10 may include one or more components that are forward-facing and one or more components that are side-facing.

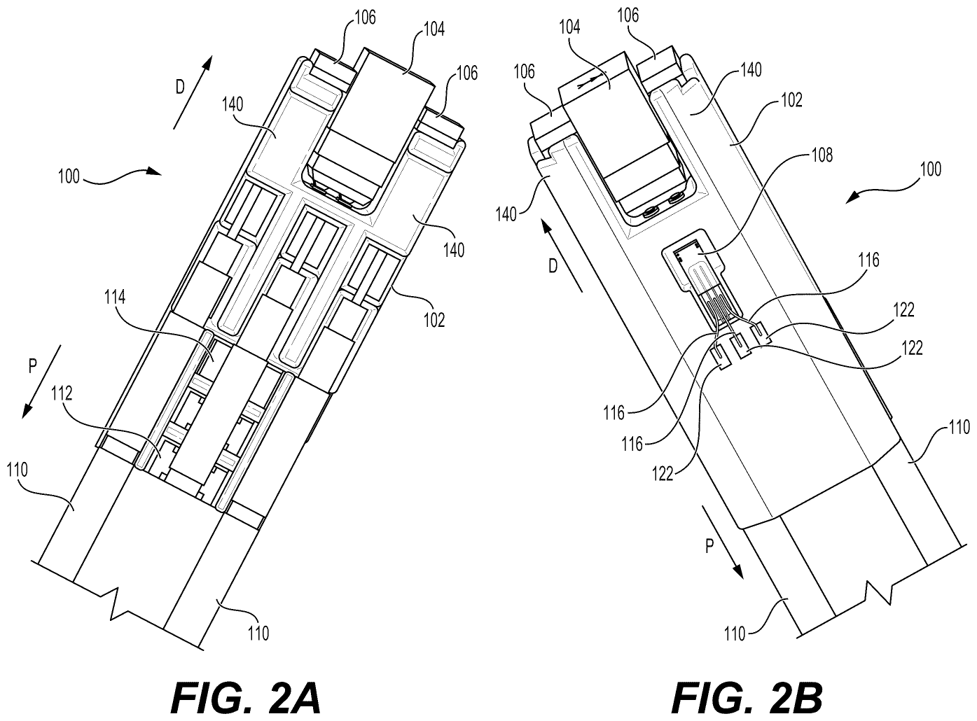

FIGS. 2A and 2B depict an exemplary electronic assembly 100, which may be disposed at least partially within distal tip 44 (FIG. 1B). FIG. 2A is a perspective view of a first side (e.g., a top side) of electronic assembly 100, and FIG. 2B is a perspective view of a second side (e.g., a bottom side) of electronic assembly 100. As discussed in detail below, various aspects or portions of electronic assembly 100 may be formed via additive manufacturing (e.g., three-dimensional printing). For example, various aspects or portions of electronic assembly 100 may include a body 102 comprising or formed of a non-conductive material or materials, such as ceramic. For example, the body 102 may comprise a 3D-printed ceramic body. Various electronic components may be coupled to (e.g., soldered onto) body 102. For example, the electronic components may include one or more imaging components to form an imager assembly. The body 102 may define a plurality of surfaces capable of supporting components. Thus, various components may be arranged on any suitable surface (e.g., any external surface) of body 102.

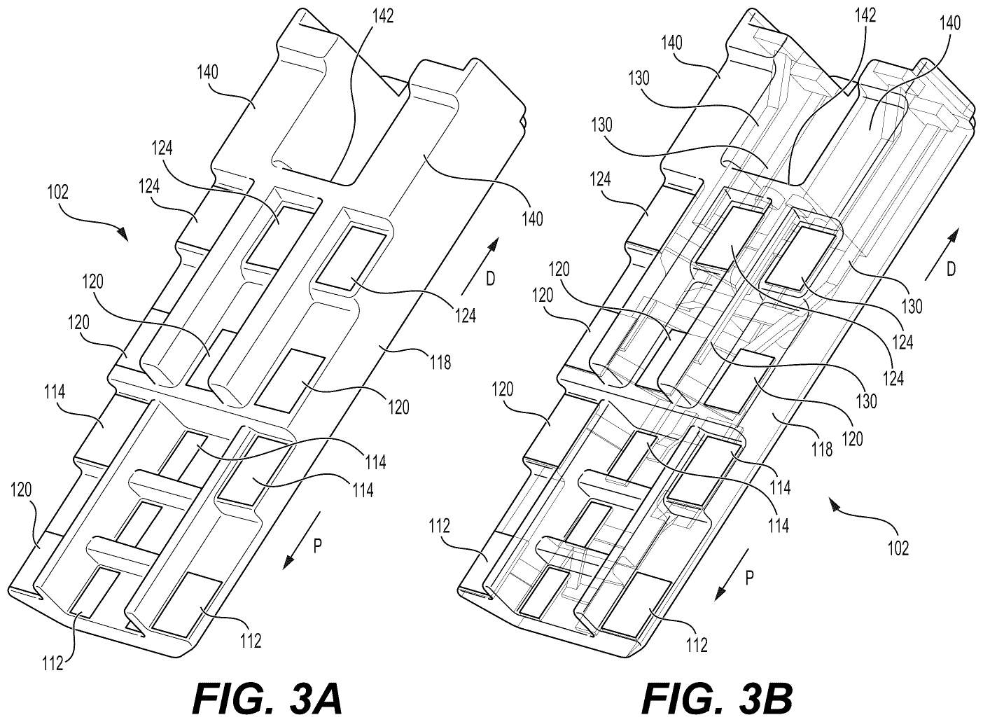

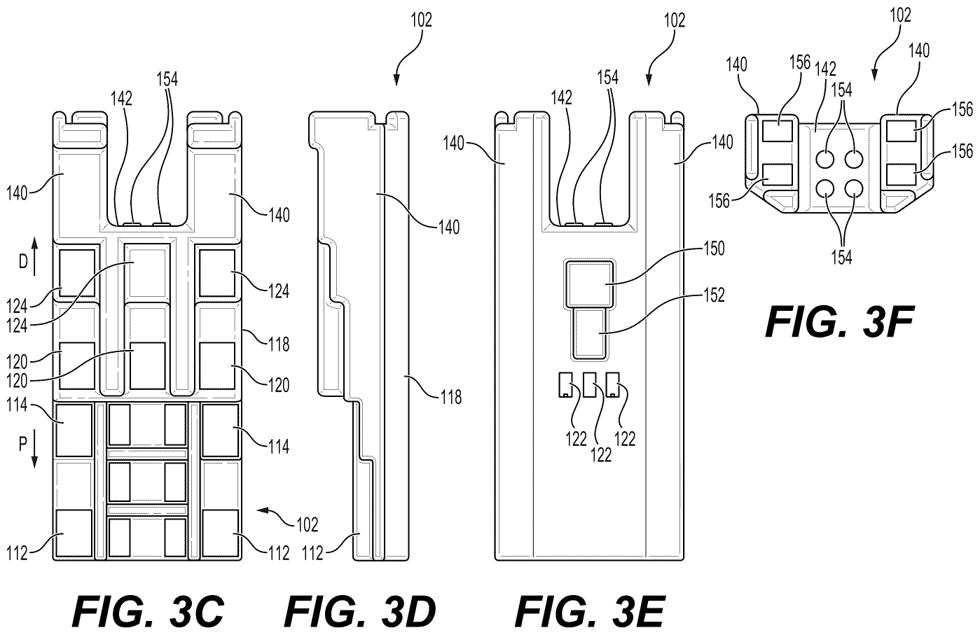

As shown in FIGS. 2A and 2B, electronic assembly 100 includes one or more imaging devices, for example, a camera 104 (e.g., corresponding to imaging device 52 in FIG. 1B) and one or more light sources or other illumination devices, for example, LEDs 106 (e.g., corresponding to light source 50 in FIG. 1B). Camera 104 and LEDs 106 may be on a distal end of electronic assembly 100. Optionally, electronic assembly 100 may include one or more sensors, for example, a pressure sensor 108, as shown. Pressure sensor 108 may be positioned on the second, bottom side of electronic assembly 100. In addition to, or in place of, pressure sensor 108, electronic assembly 100 may include one or more other components or sensors, for example, a temperature sensor. Furthermore, electronic assembly 100 may include or otherwise be coupled to one or more cables 110, for example, coaxial or micro-coaxial cables, to power and/or control the electronic components. Cables 110 may help to minimize the number of wires and/or help to provide shielding from interference (e.g., electromagnetic interference). Cables 110 may be coupled to respective electrical or cable connection points (e.g., pads or leads) 120 (FIGS. 3A and 3B), for example, via soldering.

As mentioned above, electronic assembly 100 may include one or more other electronic components, for example, associated with pressure sensor 108 or one or more other components of electronic assembly 100. For example, electronic assembly 100 may include one or more resistors 112, one or more capacitors 114, and one or more wires or bonds 116. Resistor(s) 112 and capacitor(s) 114 may be positioned on the first or top side of body 102 (FIG. 2A), and wires 116 (e.g., bond wires) may be positioned on the second or bottom side of body 102 (FIG. 2B). Wires 116 may help to connect electronic components. For example, wires 116 may provide electronic connection between pressure sensor 108 and other components of electronic assembly 100, for example, one or more pressure sensor connection points 122 (e.g., pads or leads), which may extend or otherwise face away from a surface or side (e.g., the bottom surface or side) of body 102.

As discussed in detail below, main 102 may be formed via one or more processes, including, but not limited to, multi material 3D printing or multi material insert molding. Moreover, the example depicted includes a plurality of electrical or electrically-conductive traces 130 (FIG. 3B) within body 102. Respective conductive traces may be electrically connected to respective pads (e.g., solderable pads) on various surfaces or sides of body 102. In these aspects, various components of electronic assembly 100 may be coupled to (e.g., soldered) to respective pads, and the pads and traces may electrically connect the respective components of electronic assembly 100 to proximal components (e.g., via cables 110). Moreover, the pads and/or the traces may be sized and/or spaced on or within body 102 to comply with size, shape, performance, or other desired criteria, for example, in order for electronic assembly 100 to fit within or otherwise be coupled to distal tip 44 (FIG. 2B).

Body 102 may have a size and/or shape to help support the various electronic components and/or limit or otherwise help to reduce the size of electronic assembly 100. For example, body 102 may include one or more arms 140, for example, to couple respective LEDs 106 to distal ends of one or more arms 140. In some examples, the one or more arms 140 may be adjacent to distal face 142, which may be configured to be coupled to camera 104. Body 102 may include one or more bends, contours, or other shapes or surface features that help to accommodate electronic components and/or limit or reduce the size of electronic assembly 100 in distal tip 44 (FIG. 2B).

FIGS. 3A-3F illustrate various aspects of internal or interior portions of body 102. As mentioned, body 102 may be formed via a 3D printing or additive manufacturing process and/or via a molding process. Body 102 may comprise and/or be formed (e.g., printed or molded) utilizing different materials. For example, body 102 may comprise a main or base material 118 that is electrically non-conductive or electrically insulative, such as a ceramic material. As mentioned above, body 102 may include electrical connections and elements, for example, one or more resistors 112 and/or one or more capacitors 114, for example, in a proximal portion of body 102. Such electronic elements, components, and connections may comprise an electrically conductive material or combination of materials. Additionally, body 102 may include one or more electrically conductive materials that form connection points (e.g., cable connection points 120, sensor connection points 122, and auxiliary connection points 124) and/or traces 130. Electrically conductive materials suitable for the present disclosure include, but are not limited to, copper. The connection points (e.g., cable connection points 120, sensor connection points 122, and auxiliary connection points 124) may be formed on external surfaces of body 102. Traces 130 may extend through internal or interior portions of body 102 to connect the connection points to one or more electronic components (e.g., camera 104, LED(s) 106, pressure sensor 108, etc.). Other conductive or electrical components of body 102 (e.g., resistor(s) 112, capacitor(s) 114, wires or bonds 116, etc.) may be formed in a similar manner during the manufacturing process(es).

In some examples, body 102 may be partially or entirely formed via a molding process, for example, injection molding. For example, manufacturing of body 102 may include a two-stage injection molding process. The plurality of connection points (e.g., cable connection points 120, sensor connection points 122, and auxiliary connection points 124) and/or traces 130 may inserted into or onto non-conductive base material 118 after a first or a second stage of the injection molding process. In one example, a first portion of body 102 may be formed in a first stage of the injection molding process (e.g., with base material 118), and then the plurality of traces 130 may be provided on the first portion of body 102. A second stage of the injection molding process (e.g., with base material 118) may form a second portion of body 102, for example, at least partially covering and/or insulating the plurality of traces 130. Then, the plurality of connection points (e.g., cable connection points 120, sensor connection points 122, and auxiliary connection points 124) may be provided on an exterior of body 102, with respective connection points being electrically connected to respective traces 130. Other conductive or electrical components of body 102 (e.g., resistor(s) 112, capacitor(s) 114, wires or bonds 116, etc.) may be formed in a similar manner during the manufacturing process(es).

In any of these aspects, the various connection points, traces 130, and other conductive or electronic components of body 102 may be insulated from each other by a non-conductive material or combination of materials, for example, base material 118. Base material 118 may also help to insulate the various connection points, traces 130, and/or other conductive or electronic components of body 102 (and thus electronic assembly 100) from other components of distal tip 44 (FIG. 1B).

In some aspects, cable connection points 122 may comprise electrically conductive pads configured to be electrically connected (e.g., soldered) to a sheath or outer portions of a respective cable 110 (FIG. 3A). Furthermore, auxiliary connection points 124 may comprise electrically conductive pads configured to be electrically connected (e.g., soldered) to core or inner portions of a respective cable 110 (FIG. 3A). In these aspects, auxiliary connection points 124 may be distal to cable connection points 122, allowing the core or inner portions of a respective cable 110 to extend distally of the sheath or outer portions of the corresponding cable. In some aspects, for example, as shown in FIGS. 3A and 3B, auxiliary connection points 124 may be formed on a stepped portion of body 102 relative to cable connection points 122, with the stepped portion being elevated or having a greater thickness than the more proximal portion of body 102.

FIGS. 3C-3F are different views of body 102, wherein FIG. 3C is a view of a first or top side, FIG. 3E is a view of a second or bottom side, FIG. 3D is a view of a third or lateral side (e.g., a right side relative to the orientation in FIG. 3C), and FIG. 3F is a view of a fourth or back/distal end side (e.g., the end shown as the top of FIG. 3C).

FIG. 3C illustrates body 102 as including one or more resistors 112, one or more capacitors 114, cable connection points 120, auxiliary connection points 124, etc., for example, at least partially insulated in base material 118. These electrical components may be electrically connected to respective traces 130 (FIG. 3B).

As shown in FIG. 3D, various portions of body 102 may have different thicknesses (in a top-bottom direction) and/or different widths (in a left-right direction. For example, as shown in the lateral view of FIG. 3D, a proximal end of body 102 may be thinner than a distal portion of body 102 (e.g., including arms 140). Further, for example, as shown in the distal end view of FIG. 3F, the bottom may have a tapered shape, such that the bottom side has a narrower width than the top side. The wider and/or thicker portion(s) may help to support one or more electrical or electronic components (e.g., camera 104, LED(s) 106, pressure sensor 108, etc.).

Furthermore, as shown in FIG. 3E, the second or bottom side of body 102 may include one or more mounting locations (e.g., two mounting locations 150, 152 shown), along with one or more sensor connection points 122 (e.g., three sensor connection points 122 shown). Mounting locations 150, 152 may be configured to couple one or more sensors (e.g., pressure sensor 108, a temperature sensor, etc.) to body 102. Additionally, one or more wires or bonds 116 (FIG. 2B) may connect the one or more sensors to sensor connection points 122.

Moreover, as shown, body 102 includes one or more arms 140 (e.g., two arms 140) and a distal face 142. Distal face 142 may be positioned between (e.g., laterally between) two arms 140, as shown. As shown in FIGS. 3C, 3E, and 3F, distal face 142 may include one or more imaging connection points 154, for example, four imaging connection points 154, as shown in FIG. 3F. In some aspects, the four imaging connection points 154 may be arranged in approximately a square configuration, for example, with two imaging connection points 154 arranged in a position laterally spaced from the other two imaging connection points 154. Imaging connection point(s) 154 may comprise pads or leads configured to be electrically and/or mechanically coupled (e.g., soldered) to one or more imaging devices (e.g., camera 104). Imaging connection point(s) 154 may be electrically connected to one or more traces 130. The one or more traces 130 may convey power and/or signals (e.g., on/off, image capture settings, etc.) from one or more cables 110 distally to the one or more imaging devices. The one or more traces 130 may also convey signals (e.g., image signals, video signals, etc.) from the one or more imaging devices proximally to one or more cables 110 (e.g., through body 102).

As shown in FIG. 3F, arm(s) 140 may include one or more illumination connection points 156. For example, each arm 140 may include two illumination connection points 156 on a distal portion or distal face of the arm 140 (e.g., the two illumination connection points 156 spaced apart vertically). Illumination connection point(s) 156 may comprise pads or leads configured to be electrically and/or mechanically coupled (e.g., soldered) to one or more illumination devices (e.g., LED(s) 106). Moreover, illumination connection point(s) 156 may be electrically connected to one or more traces 130. In this aspect, the one or more traces 130 may convey power and/or signals (on or off, illumination intensity, etc.) distally from one or more cables 110 to the one or more illumination devices (e.g., LED(s) 106). Furthermore, the one or more traces 130 may convey signals (fault signals, etc.) proximally from the one or more illumination devices (e.g., LED(s) 106) to one or more cables 110 (e.g., through body 102).

FIG. 4 illustrates a perspective view of another example according to the present disclosure, showing distal tip 234 of a shaft 232 of a medical device, for example, of insertion portion 14 of medical device 10 shown in FIGS. 1A and 1B. Shaft 232 may include any of the features of shaft 42 discussed above. As shown, distal tip 234 includes one or more imaging devices (e.g., a camera 204) and one or more illumination devices (e.g., one or more LED(s) 206), for example, on a distalmost face 246 of distal tip 234. Additionally, distal tip 234 includes a working channel opening 248, for example, which connects to a lumen within shaft 232. Moreover, insertion portion 14, in this example, may include an articulation joint 264 (alternatively referred to as articulation portion 264), for example, at a distal portion of insertion portion 14 that is proximal of distal tip 234. In some aspects, a control or actuation mechanism (e.g., similar to lever 22 in FIG. 1A) may control the deflection, articulation, or movement of a distal portion of insertion portion 14 via articulation joint 264.

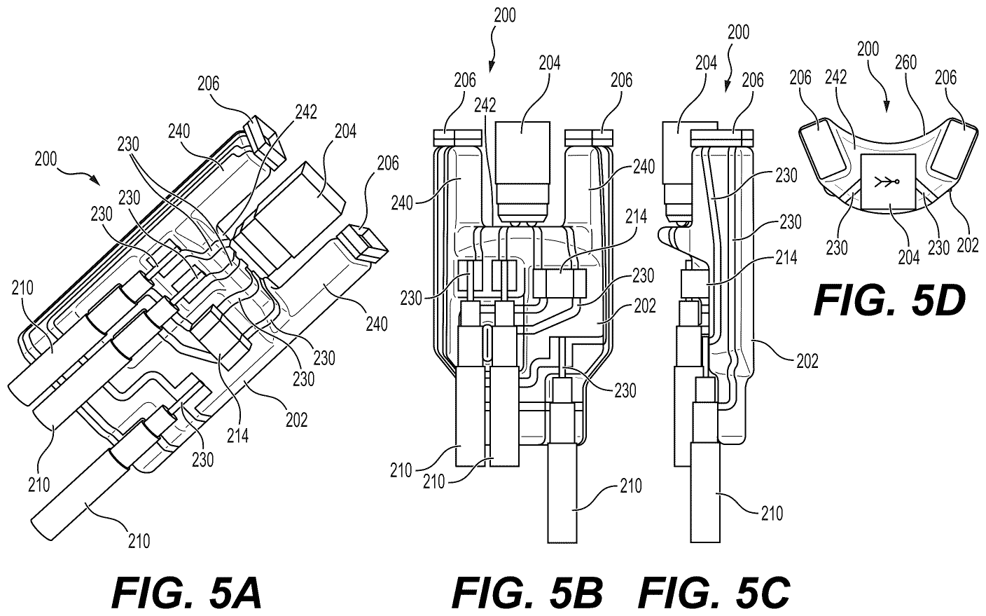

FIGS. 5A-5D are various views of another exemplary electronic assembly 200, according to this disclosure, which may be disposed at least partially within a distal tip of a medical device, for example, distal tip 44 of medical device 10 (FIG. 1B). FIG. 5A is a perspective view and FIG. 5B is a plan view of a first side (e.g., a top side) of electronic assembly 200. FIG. 5C is a side view of a lateral side (e.g., the right side in FIGS. 5A and 5B) of electronic assembly 200, and FIG. 5D is a distal end view of electronic assembly 200. As discussed in detail below, various aspects or portions of electronic assembly 200 may be formed via additive manufacturing (e.g., three-dimensional printing) or molding. For example, various aspects or portions of electronic assembly 200 may include a body 202, for example, formed of or comprising a non-conductive material, such as a ceramic. As discussed below, various electrical connections and/or components may be coupled to (e.g., soldered onto) body 202. When the components include imaging devices, for example, the electronic assembly 200 may form an imager assembly. Various components may be arranged on any suitable surface of body 202, compared to a printed circuit board that can only accommodate components on top and bottom surfaces.

Electronic assembly 200 includes body 202 and a plurality of conductive components and/or electronic/electrical elements. As discussed above, electronic assembly 200 may include one or more imaging devices, for example, camera 204, and one or more illumination devices, for example, LED(s) 206. Camera 204 may be coupled to a distal face 242 of body 202, and LEDs 206 may be coupled to respective arms 240. Arms 240 may be positioned on opposing sides (e.g., lateral sides) of distal face 242. In some aspects, as shown in FIG. 5D, a distal portion of body 202 may include a curved inner face 260. Curved inner face 260 may help body 202 to be positioned adjacent to one or more lumens or working channels (e.g., working channel opening 248 in FIG. 4), providing space for a relatively larger size of the one or more lumens or working channels and/or providing for a relatively smaller overall size of distal tip 234.

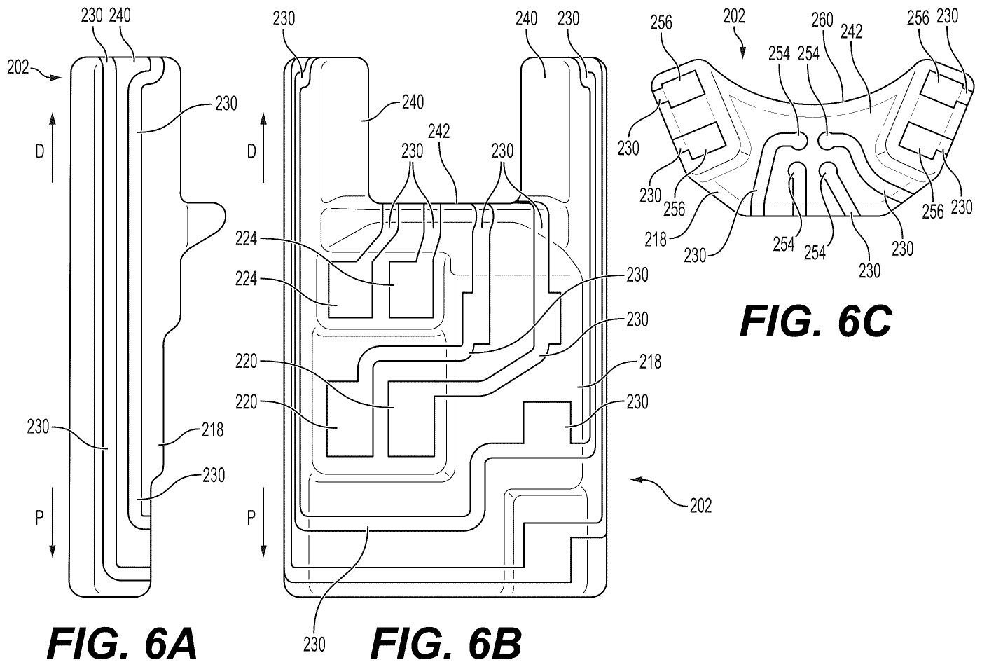

Electronic assembly 200 may include any of the features of electronic assembly 100. For example, electronic assembly 200 may include one or more sensors, for example, a pressure sensor and/or a temperature sensor. Electronic assembly 200 may also include one or more resistors and/or one or more capacitors 214, as discussed above in connection to electronic assembly 100. The pressure sensor and/or the temperature sensor may be positioned on a second, bottom side of electronic assembly 200, or on another portion of electronic assembly 200. Furthermore, as discussed above, electronic assembly 200 may include or otherwise be coupled to one or more cables 210, for example, coaxial or micro-coaxial cables. Cables 210 may be coupled to respective cable connection points 220 (e.g., pads or leads) (FIG. 6B), for example, via soldering. Moreover, cables 210 may couple electrically and/or communicatively one or more components of electronic assembly 200 to one or more proximal elements (e.g., controllers, processors, displays, etc.). As discussed in detail below, cables 210 may be coupled electrically and/or communicatively to one or more components of electronic assembly 200 via one or more respective traces 230 formed in an exterior of electronic assembly 200.

Additionally, electronic assembly 200 may include one or more other electronic components, for example, associated with the pressure sensor or one or more other components of electronic assembly. For example, electronic assembly 200 may include one or more resistors, one or more capacitors, and one or more wires or bonds, etc., as discussed above in connection to electronic assembly 100.

As mentioned above with respect to body 102, body 202 may be formed via one or more processes, including but not limited to, multi material 3D printing, multi material insert molding (e.g., plastic injection molding), laser direct structuring, or machining. A plurality of electrically conductive traces 230 are positioned on various external portions of body 202. Respective conductive traces 230 may be electrically connected to respective pads (e.g., solderable pads) on various surfaces or sides of body 202. In these aspects, various components of electronic assembly 200 may be coupled to (e.g., soldered) to respective pads, and the pads and traces may electrically connect the respective components of electronic assembly 200 to proximal components (e.g., via cables 210). The pads and/or the traces may be sized and/or spaced on or within body 202 to satisfy desired size, shape, performance, or other criteria to allow for electronic assembly 200 to fit within or otherwise be coupled to distal tip 44 or distal tip 234 (FIGS. 1B and 4).

Body 202 may have a size and/or shape to help support the various electronic components and/or to help to reduce the size of electronic assembly 200. For example, body 202 may include one or more arms 240 to couple respective LEDs 206 to distal ends of the one or more arms. Additionally, the one or more arms 240 may be adjacent to a distal face 242, which may be configured to be coupled to camera 204. Moreover, body 202 may include one or more bends, contours, or other shapes or surface features that help to accommodate the electronic components and/or reduce the size of electronic assembly 200 in distal tip 44 or distal tip 234 (FIGS. 1B and 4).

FIGS. 6A-6C illustrate various aspects of body 202 without various electronic components (e.g., camera 204, LED(s) 206, cables 210, etc.) but including electrical connections (e.g., traces 230, cable connection points 220, etc.). FIG. 6A is a side view of body 202, for example, the left side relative to the arrangement shown in FIGS. 5A and 5B). FIG. 6B is a top view of body 202, and FIG. 6C is a distal end view of body 202 (e.g., including curved inner face 260).

Body 202 comprises a base material 218, for example, a non-conductive and/or insulating material, e.g., a ceramic. Additionally, body 202 includes plurality of traces 230, one or more cable connection points 220, one or more sensor connection points (not shown), and one or more auxiliary connection points 224, for example, on the top side of body 202. As shown in FIGS. 6A and 6B, body 202 may include different thicknesses (e.g., features like stepped portions with differing heights or levels). For example, a proximal end or portion may be thinner (see FIG. 6A) than an intermediate portion and/or a distal portion of body 202. The heights, levels, or steps may help to maintain cable(s) 210 in straight configuration(s) as various portions of cable(s) 210 are coupled to body 202. The size and/or shape of body 202 may also help to ensure proper placement of cable(s) 210 relative to body 202 and/or ensure proper contact between various portions of cable(s) 210) with respective portions of body 202 (e.g., reducing the potential for stresses on the couplings/solder joints, improving couplings/solderability, etc.).

In some aspects, as shown in FIG. 6A, body 202 may include a wall or barrier between proximal portions (e.g., the various connection points, cable(s) 210, etc.) and the distal portions (e.g., imaging connection points 254, illumination connection points 256, etc.). The wall or barrier may help in the alignment and/or positioning of cable(s) 210, may help to prevent shorts (e.g., solder shorts) between wires or portions of electronic assembly 200, help to allow for consistent and/or accurate placement of wires on body 202, help to reduce the overall space required for electronic assembly 200, etc. Additionally, the shape of body 202 may help to recess portions of cable(s) 210, for example, toward the center of body 202. The shape of body 202 and/or cable(s) 210 may also help to provide additional space inside the shaft of the medical device (e.g., shaft 232) and/or help to keep wires or other conductive elements away from the exterior of the medical device (e.g., away from the exterior of insertion portion 14). As a result, shaft 232 may exhibit lower stresses and/or strains when distal tip 234 is articulated or otherwise maneuvered. Body 102 of electronic assembly 100 (e.g., as shown in FIGS. 2A, 2B, and 3A-3F) may include one or more of the aforementioned shapes and/or benefits of body 202.

Furthermore, body 202 may include a plurality of imaging connection points 254 (e.g., on distal end face 242) and a plurality of illumination connection points 256 (e.g., on arm(s) 240). In some aspects, as shown in FIG. 6C, imaging connection points 254 and/or illumination connection points 256 may be continuations or otherwise continuously formed with respective traces 230. In some aspects, body 202 may include one or more sensor mounting locations and/or bonds, as discussed in connection to body 102 above. Traces 230 may electrically and/or communicatively couple various connection points (e.g., cable connection points 220, the sensor connection points, auxiliary connection points 224) to one or more other connection points (e.g., the sensor mounting locations or bonds, imaging connection point(s) 254, and/or illumination connection point(s) 256), as discussed above. The electrical and/or communicative coupling may be one way (e.g., proximal to distal or distal to proximal) or may be two way (e.g., proximal to distal and distal to proximal). Moreover, base material 218 may help to insulate traces 230 and/or the various connection points from each other. In some aspects, base material 218 may also help to insulate the various connection points, traces 230, and/or other conductive or electronic components of body 202 (and thus electronic assembly 200) from other components of distal tip 44 or distal tip 234 (FIGS. 1B and 4).

Various aspects of this disclosure may help to reduce the overall space sufficient for various electronic components (e.g., cameras or other imaging devices, LEDs or other illumination devices, pressure or temperature sensors, etc.) in a distal tip of a medical device (e.g., endoscope, ureteroscope, etc.). Additionally, various aspects of this disclosure may help to allocate space to allow for a larger working channel or larger number of working channels or lumens (e.g., irrigation, suction, etc.) in an insertion portion of the medical device. In some aspects, the LED(s) or illumination device(s) may be extended upward, for example, toward the top or an end portion of the camera(s) or imaging device(s). The shape of body 102, 202 (e.g., arm(s) 140, 240) may help to orient or otherwise position the LED(s) or other illumination device(s) relative to the camera(s) or other imaging device(s). This position of the LED(s) or other illumination device(s) may be recessed from the camera(s), which may help to minimize patient risk and/or potential light leakage from the LED(s) or other illumination device(s) into the camera(s).

One or more portions of the electronic assembly (e.g., electronic assembly 100, 200 may be overmolded with a material or combination of materials (e.g., an epoxy), for example, to help prevent contact with the subject, also reducing a need for biocompatibility testing. For example, one or more LED(s) 106, 206 may be overmolded with epoxy or other material that allows for the passage of light therethrough while providing a barrier between the LED(s) 106, 206 and the subject. In some aspects, capacitor(s) 114, 214 may also help to provide voltage decoupling on various aspects of electronic assemblies 100, 200, for example, to help improve image quality from camera(s) 104, 204. Capacitor(s) 114, 214, resistor(s) 112, and/or other electronic components may also be recessed toward a center of body 102, 202, for example, to help minimize space. In some aspects, one or more sensors (e.g., pressure sensor(s) 108, 208) may form or be a part of a circuit, for example a Wheatstone bridge, formed within or as a part of electronic assembly 100, 200. Positioning the circuit (e.g., the Wheatstone bridge) within or as a part of electronic assembly 100, 200, with electronic assembly 100, 200 in distal tip 44, 234 (FIGS. 1B and 4) may help to avoid the need for long and/or thin cables extending from distal tip 44, 234 proximally though the medical device (e.g., to a circuit board in handle portion 12). In some aspects, positioning at least a portion of the circuit within or as a part of electronic assembly 100, 200, with electronic assembly 100, 200 in distal tip 44, 234 (FIGS. 1B and 4) may help to reduce the total number of cables or wires that extend from distal tip 44, 234 proximally through the medical device (e.g., to a circuit board in handle portion 12). As such, there may be a reduced likelihood of the connections between distal tip 44, 234 and handle portion 12 breaking, requiring voltage stabilization, etc.

While principles of this disclosure are described herein with the reference to illustrative examples in a particular context and for particular medical procedures, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

1. An electronic assembly for a medical device, the electronic assembly including:
a body including a plurality of electrical connection points and a plurality of electrical traces in a material that is electrically insulative, wherein the plurality of electrical connection points are on one or more external surfaces of the body, wherein the plurality of electrical connection points includes proximal connection points and distal connection points, and wherein each electrical trace of the plurality of electrical traces connects a proximal connection point to a respective distal connection point;
at least one imaging device coupled to one or more of the distal connection points; and
at least one light source coupled to one or more of the distal connection points.

2. The electronic assembly of claim 1, wherein the at least one imaging device and the at least one light source face in a same direction.

3. The electronic assembly of claim 1, wherein the plurality of electrical connection points includes a plurality of cable connection points and at least one sensor connection point.

4. The electronic assembly of claim 1, further comprising at least one sensor coupled to the body on a side of the body opposite at least one electrical connection point of the plurality of electrical connection points.

5. The electronic assembly of claim 4, wherein the at least one sensor includes a pressure sensor.

6. The electronic assembly of claim 1, wherein the body includes an end face and at least one arm adjacent to, and extending distally of, the end face.

7. The electronic assembly of claim 6, wherein the end face includes at least one of the distal connection points, and the at least one imaging device is coupled to the distal connection point on the end face.

8. The electronic assembly of claim 6, wherein the at least one arm includes at least one of the distal connection points, and the at least one light source is coupled to the distal connection point on the at least one arm.

9. The electronic assembly of claim 1, wherein the at least one light source includes two light-emitting diodes (LEDs) positioned on opposing sides of the at least one imaging device.

10. The electronic assembly of claim 9, wherein the LEDs are proximal to the at least one imaging device.

11. The electronic assembly of claim 1, wherein the material of the body comprises a ceramic and/or other non-conductive materials.

12. The electronic assembly of claim 1, wherein the plurality of electrical traces comprise copper and/or other conductive materials.

13. The electronic assembly of claim 1, wherein the plurality of electrical traces passes through an interior of the body.

14. The electronic assembly of claim 1, further comprising a capacitor and a resistor.

15. The electronic assembly of claim 1, wherein the body includes an end face that has a curved surface.

16. An electronic assembly for a medical device, the electronic assembly including:
a body that includes an end face and at least one arm adjacent to and extending distally of the end face, wherein the body comprises an electrically insulative material, a plurality of electrical connection points, and a plurality of electrically conductive traces;
at least one imaging device coupled to the end face of the body; and
at least one light source coupled to the at least one arm.

17. The electronic assembly of claim 16, wherein the plurality of electrical connection points includes proximal connection points and distal connection points, and wherein each electrically conductive trace of the plurality of electrically conductive traces electrically connects a proximal connection point to a respective distal connection point.

18. The electronic assembly of claim 16, further comprising a pressure sensor coupled to one or more of the electrical connection points of the plurality of electrical connection points.

19. An electronic assembly for a medical device, the electronic assembly including:
a body that includes an end face and at least one arm adjacent to the end face, wherein the body comprises an electrically insulative material, a plurality of electrical connection points on an external surface of the body, and a plurality of electrically conductive traces that passes through an interior of the body between two electrical connection points of the plurality of electrical connection points;
at least one imaging device coupled to the end face; and
at least one light source coupled to the at least one arm.

20. The electronic assembly of claim 19, wherein the plurality of electrical connection points includes proximal connection points and distal connection points, a proximal portion of the body including the proximal connection points and an intermediate portion of the body including the distal connection points, and wherein the body includes a stepped portion between the proximal portion and the intermediate portion.
